(19) United States
(12) Patent Application Publication
(10) Pub. No.: US 2009/0286030 A1
(43) Pub. Date: Nov. 19, 2009

(54) INSTRUMENT HOLDER AND CONNECTOR

(75) Inventors: Maxime Robert, L'Ancienne-Lorette (CA); Ghislain Parent, Saint-Isidore (CA)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 12/465,101

(22) Filed: May 13, 2009

Related U.S. Application Data
(60) Provisional application No. 61/053,791, filed on May 16, 2008.

(51) Int. Cl. B32B 1/08

(52) U.S. Cl. 428/36.91

(57) ABSTRACT

A pressure/flow regulator includes a device having an input end that is connectable to a source of fluid, and an output end that is fluidly connected to a medical instrument. The device has a passage running from the input end to the output end to carry the fluid therethrough. A polymeric sleeve is disposed on the device. The polymeric sleeve covers at least one aperture that communicates with the passage. The polymeric sleeve has a resiliency capable of retaining the fluid within the at least one aperture until a predetermined pressure of the fluid within the aperture is attained at which point the sleeve deforms releasing pressure of the fluid within the device and preventing pressure damage to the medical instrument.

### RELATED APPLICATIONS

This application claims the benefit of U.S. Provisional Application No. 61/053,791, filed May 16, 2008, which is fully incorporated herein by reference.

### FIELD OF THE INVENTION

The present invention relates generally to the field of cleaning and disinfecting devices with narrow lumens, and more particularly to cleaning and disinfecting medical instruments, such as minimally invasive (MIS) instruments.

### BACKGROUND OF THE INVENTION

Fluid microbial cleaning and disinfecting systems are typically designed to cause microbes on an item to be removed or disinfected by a fluid anti-microbial agent. Such systems operate in a variety of ways, including spraying the item(s) with the anti-microbial solution, immersing an item(s) in an anti-microbial solution, surrounding the item(s) with anti-microbial vapor and the like. It has been proposed that one way to clean lumens in medical instruments is to force the anti-microbial solution through the internal lumens of the instrument. In this respect, the anti-microbial solution is supplied to the medical instrument under pressure which is sufficient to force the solution through the lumens and passageways of the medical instrument. In cleaning medical instruments in such a fashion, it is important that the pressure of the anti-microbial solution be maintained at a pressure below that which could damage delicate components within the medical instrument. Operating at pressures above the original equipment manufacturer (OEM) safe recommended levels could damage the devices. Systems that restrict flow or direct flow through narrow passages often increase the actual pressure exerted on the medical instruments.

It is known to microbially disinfect medical instruments and equipment in washers by exposing the medical instruments or equipment to a liquid anti-microbial solution. Known washing systems for microbial disinfection of medical instruments and equipment typically include baskets or racks that retain the various equipment or instruments to be cleaned. The baskets or racks are usually movable within the washer to facilitate loading and unloading of the washer. It is also known to have spray heads mounted to the movable racks or baskets to direct the anti-microbial solution over the instruments therein. Some type of fluid connection is therefore required between a stationary fluid inlet line on the washer and a movable fluid line on the rack or basket, or between the stationary fluid inlet line on the washer and some accessory used within the washer.

The present invention overcomes these and other problems, and provides a pressure/flow regulator that regulates pressure and flow to a medical instrument during a sterilization procedure.

### SUMMARY OF THE INVENTION

In accordance with one aspect of the present invention, there is provided a pressure/flow regulator that includes a device having an input end that is connectable to a source of fluid, and an output end that is fluidly connected to a medical instrument. A passage runs from the input end to the output end to carry the fluid therethrough. A polymeric sleeve is disposed on the device. The polymeric sleeve covers at least one aperture that communicates with the passage. The polymeric sleeve has a resiliency capable of retaining the fluid within the at least one aperture until a predetermined pressure of the fluid within the aperture is attained at which point the sleeve deforms releasing pressure of the fluid within the device and preventing pressure damage to the medical instrument.

One advantage of the present invention is a device that prevents damage to sensitive medical instruments in a microbial deactivating reprocessor.

Another advantage is a device as described above that regulates the operating pressure applied to a medical instrument in a microbial deactivating system.

Another advantage of the present invention is a fluid connection that is quickly and easily connected and disconnected.

Another advantage of the present invention is a fluid connection as described above for use in a washer for microbially cleaning and disinfecting items.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a partially sectioned, perspective view of a washer rack within a washing chamber showing a flexible connector attached to a canulated surgical instrument;

FIG. 2 is an enlarged view of the flexible connector shown in FIG. 1;

FIG. 3 is an enlarged cross-sectional view of a portion of the flexible connector showing a pressure/flow regulator connected to a portion of tubing forming the rack shown in FIG. 1;

FIG. 4 is a cross-sectional view taken along lines 4-4 of FIG. 3;

FIG. 5 is an enlarged cross-sectional view of the pressure/flow regulator illustrating the operation thereof;

FIG. 6 is an exploded, perspective view of pressure/flow regulator of the present invention;

FIG. 7 is an enlarged view of an instrument connector attached to one end of the flexible connector, showing the instrument connector attached to a tubular portion of a canulated surgical instrument; and

FIG. 8 is a sectional view of the instrument connector shown in FIG. 7 without the tubular portion of the canulated surgical instrument attached thereto.

### DETAILED DESCRIPTION OF PREFERRED EMBODIMENT

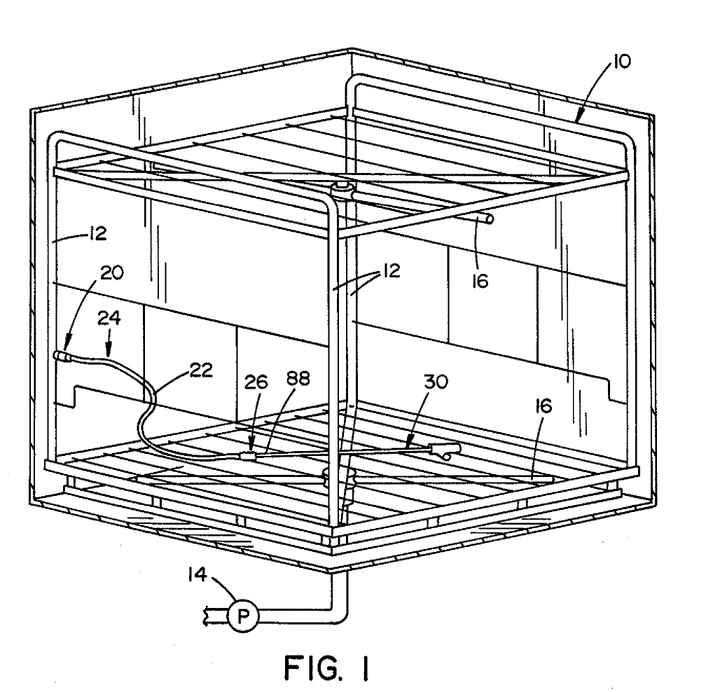

Referring now to the drawings wherein the showings are for the purpose of illustrating a preferred embodiment of the invention only, and not for the purpose of limiting same, FIG. 1 shows a rack 10 for use in a medical washer. In FIG. 1, walls of a medical washer are shown for illustrative purposes. Rack 10 for use in a medical washer would typically be movable into and out of a washing chamber. In the embodiment shown, rack 10 is formed of hollow, metal tubing 12. Connection means (not shown) on the lower portion of rack 10 communicate with a fluid pump 14 for conveying fluid through tubular portions of rack 10 to spray arms 16 on rack 10. Spray arms 16 are designed to convey washing and rinsing fluids onto objects such as medical devices supported by rack 10.

In the embodiment shown, rack 10 is designed to include a flexible connector 24 for cleaning canulated surgical instruments 30.

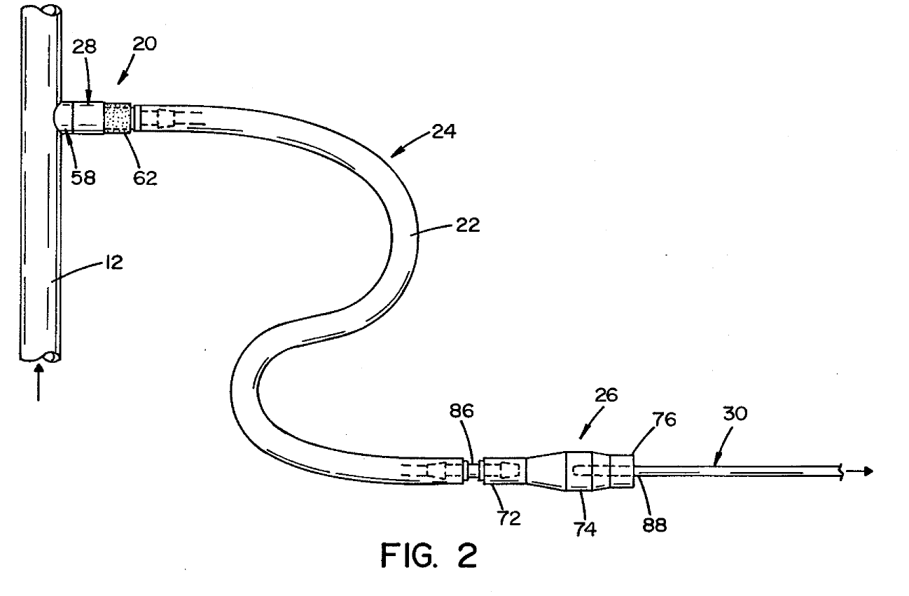

Flexible connector 24, best seen in FIG. 2, is comprised of a flexible tubing 22 having a pressure/flow regulator 20 attached to one end and an instrument connector 26 attached to the other end. One end of pressure/flow regulator 20 is attachable to a portion of hollow metal tubing 12 to facilitate a flow of cleaning fluids and rinsing fluids through flexible connector 24 to a tubular piece or portion of canulated surgical instrument 30, as illustrated in FIG. 2.

As indicated above, flexible connector 24 is generally comprised of flexible tubing 22, pressure/flow regulator 20 and instrument connector 26. Flexible tubing 22 is comprised of medical grade plastic tubing, such as by way of example and not limitation, polyethylene, polypropylene or TEFLON®.

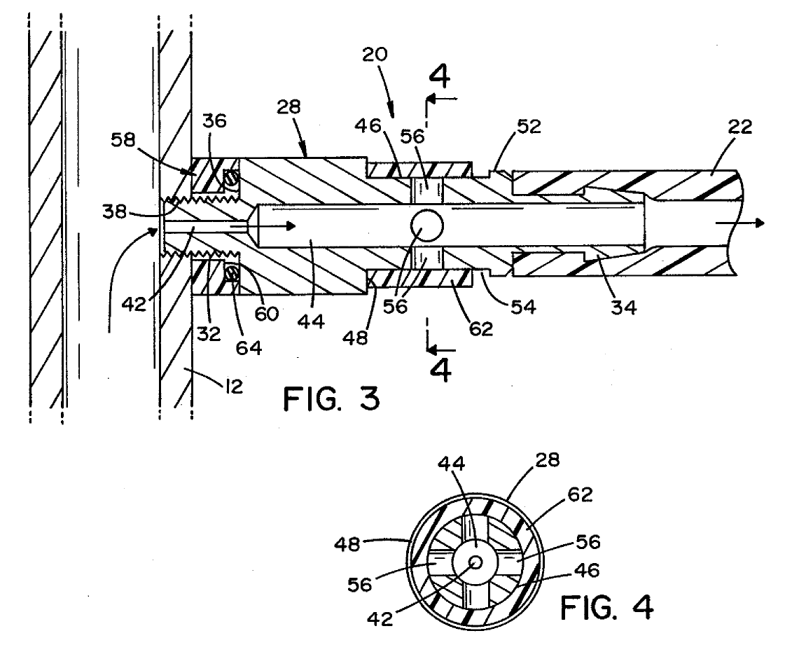

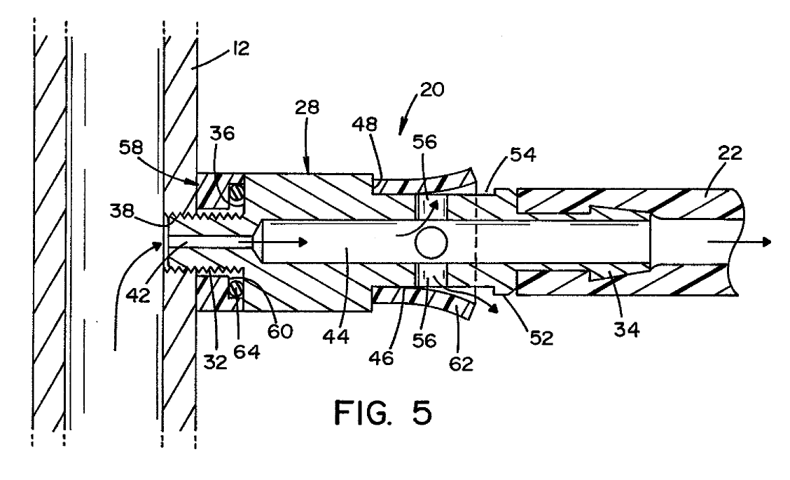

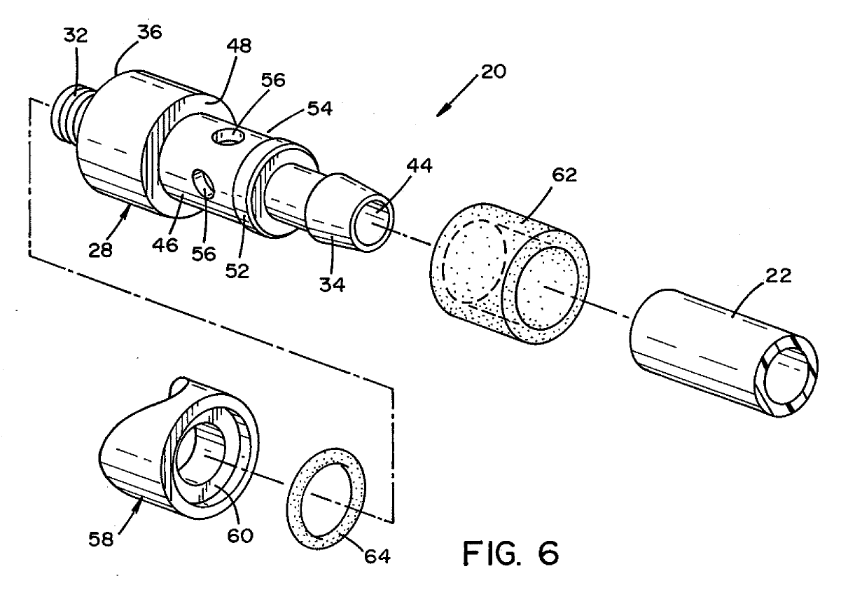

Referring now to FIGS. 3-6, pressure/flow regulator 20 is best seen. Pressure/flow regulator 20 includes a body portion 28 having a threaded fastener portion 32 at one end and a barbed tube connecting portion 34 at the other end. As best seen in FIGS. 3 and 5, threaded fastener portion 32 extends from an annular planar surface 36 of body portion 28. Threaded fastener portion 32 is dimensioned to be received in threaded opening 38 in a portion of hollow metal tubing 12 forming rack 10. An orifice 42 extends axially through threaded fastener portion 32 of pressure/flow regulator 24. Orifice 42 communicates with a large passage 44 that extends through body portion 28 and barbed tube connecting portion 34 at the other end of body portion 28. Body portion 28 of pressure/flow regulator 20 includes a cylindrical portion 46. In the embodiment shown, the entire pressure/flow regulator 20 is generally cylindrical in shape. Cylindrical portion 46 of body portion 28 has a generally reduced diameter relative to the remaining portion of body portion 28 so as to define a corner or shoulder 48 where cylindrical portion 46 extends from body portion 28. An outwardly extending flange or collar 52 is formed at one end of cylindrical portion 46 to define a cylindrical recess 54 about body portion 28. One edge of annular flange 52 is champhered. Barbed tube connecting portion 34 extends from cylindrical portion 46 of pressure/flow regulator 20. In this respect, the barbed end of pressure/flow regulator 20 defines a male fitting having a barbed end dimensioned to receive flexible tubing 22, as illustrated in FIGS. 3 and 5. At least one aperture 56 extends radially from large passage 44 through cylindrical portion 46 of pressure/flow regulator 20. In the embodiment shown, four equally spaced apertures 56 extend through cylindrical portion 46 such that large passage 44 through body portion 28 of pressure/flow regulator 24 communicates with the exterior thereof.

In accordance with one aspect of the present invention, a resilient, flexible sleeve is provided to fit onto cylindrical portion 46 of pressure/flow regulator 20, as best seen in FIGS. 3-5. Sleeve 62 is generally formed of an elastomeric material. By way of example and not limitation, sleeve 62 is formed of EPDM rubber (ethylene propylene diene monomer rubber). Sleeve 62 is designed to snuggly fit onto cylindrical portion 46 of pressure/flow regulator 20 with sleeve 62 covering apertures 56 formed therein. In this respect, the material forming sleeve 62 is compounded to meet specific properties, as shall be described in greater detail below.

In the embodiment shown, pressure/flow regulator 20 is designed to be attached to a portion of hollow metal tubing 12 forming rack 10 such that the passageway through hollow metal tubing 12 is in communication with orifice 42 and large passage 44 extending through pressure/flow regulator 20. In this respect, threaded fastener portion 32 of pressure/flow regulator 20 is threaded into hollow metal tubing 12. A spacer 58, as seen in FIGS. 3 and 5, is disposed between body portion 28 of pressure/flow regulator 20 and hollow metal tubing 12. One end of spacer 58 is formed to have a concave, cylindrical surface. The cylindrical surface is dimensioned to conform to the curvature of hollow metal tubing 12 of rack 10 wherein spacer 58 abuts and mates with the surface of hollow metal tubing 12. The other face of spacer 58 is flat to abut with annular planar surface 36 of body portion 28 of pressure/flow regulator 20. A cylindrical recess 60, or counter bore, is formed in annular planar surface of spacer 58 to receive an O-ring 64. O-ring 64 is dimensioned to be compressed between body portion 28 of pressure/flow regulator 20 and spacer 58 when pressure/flow regulator 20 is threaded into hollow metal tubing 12, as best seen in FIGS. 3 and 5.

Sleeve 62 is placed onto cylindrical portion 46 of pressure/flow regulator 20 by sliding sleeve 62 over barbed tube connecting portion 34 and cylindrical portion 46 on the end of body portion 46. The champhering of barbed tube connecting portion 34 facilitates positioning sleeve 62 onto cylindrical portion 46 of pressure/flow regulator 20. In a conventionally known manner, flexible tubing 22 is forced onto barbed tube connecting portion 34 of pressure/flow regulator 20 to secure flexible tubing 22 to the end of pressure/flow regulator 20.

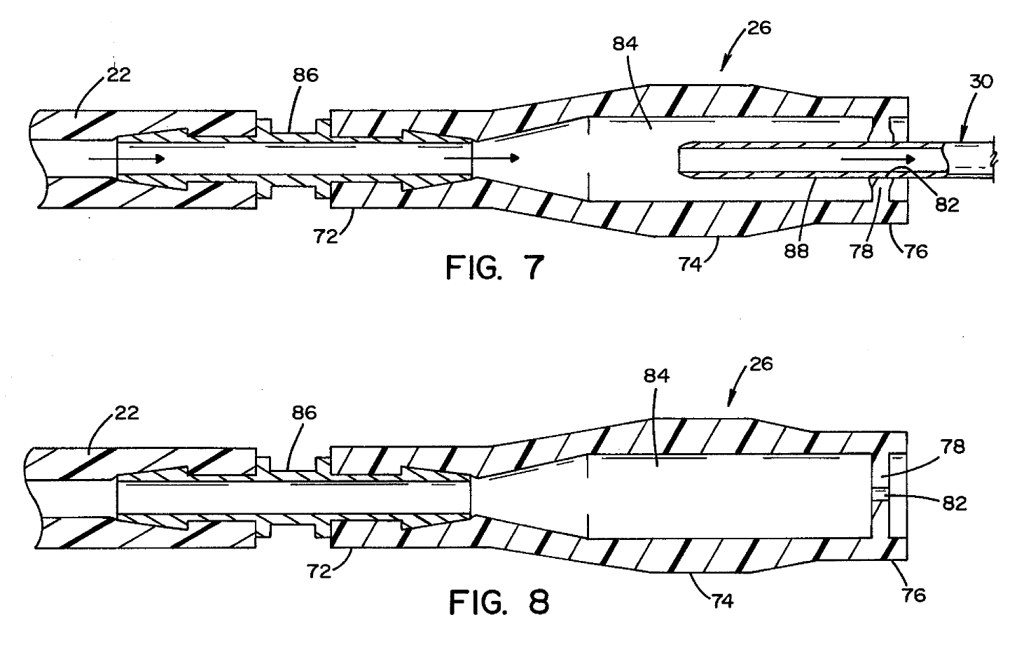

Referring now to FIGS. 7 and 8, instrument connector 26 is best seen. Instrument connector 26 is generally tubular in shape and is formed of a resilient elastomeric material. Instrument connector 26 has a first end 72 generally conforming in size and dimension of flexible tubing 22, an enlarged and reinforced intermediate portion 74 and an instrument connection end 76. Instrument connector end 76 of instrument connector 26 includes a generally transverse wall 78 integrally molded as part of instrument connector 26. A transverse wall aperture 82 extends through transverse wall 78. As illustrated in FIG. 8, intermediate portion 74 and instrument connector end 76 of instrument connector 26 define a cavity 84 larger than the passage through flexible tubing 22 and first end 72 of instrument connector 26. Instrument connector 26 is integrally molded of a resilient polymer material. Instrument connector 26 is designed to be attached to flexible tubing 22 by a double end male fitting tube connector 86, as illustrated in FIGS. 7 and 8. Transverse wall aperture 82 in instrument connector 26 is dimensioned to receive a tubular portion 88 of canulated surgical instrument 30, as illustrated in FIG. 7.

Referring to the operation of the present invention, threaded fastener portion 32 of pressure/flow regulator 20 is mounted to hollow metal tubing 12 of rack 10. Instrument connector end 76 of flexible connector 24 is attached to tubular portion 88 of canulated surgical instrument 30 by forcing instrument connector end 76 of instrument connector 26 over tubular end portion 88 of canulated surgical instrument 30. As indicated in FIG. 7, transverse wall 78 on instrument connector end 76 of instrument connector 26 forms a seal around tubular portion 88 of canulated surgical instrument 30. Canulated surgical instrument 30 is then placed into rack 10. Rack 10 is then inserted into the washing chamber. During a washing cycle, washing fluids and rinse fluids are sequentially forced through hollow metal tubing 12 of rack 10 to spray arms 16 and nozzles attached thereto. The washing and rinse fluids wash and rinse the exterior of medical instruments as is conventionally known. The washing and rinse fluids are also forced through hollow metal tubing 12, through pressure/flow regulator 20 and into flexible tubing 22 connected to canulated surgical instrument 30. Pressure/flow regulator 20, connected to flexible tubing 22, limits the flow and pressure to canulated surgical instrument 30 to prevent damage thereto. In this respect, orifice 42 in pressure/flow regulator 20 limits flow into flexible connector 24. The pressure within flexible connector 24 is regulated by apertures 56 in cylindrical portion 46 of pressure/flow regulator 20 and sleeve 62. As illustrated in FIG. 5, sleeve 62 is designed to have a certain resilience and to flex when the pressure on the inner surface thereof exceeds the designed parameters of sleeve 62. As illustrated in FIG. 5, if the pressure should exceed a desired value, for example, about 10 psig to about 15 psig for many canulated instruments, the pressure would cause sleeve 62 to deform, separate from pressure/flow regulator 20 and allow fluid to escape therefrom. The fluid escaping from pressure/flow regulator 20 is collected within the washing chamber during a washing or rinsing cycle. Thus, the fluid flow to canulated medical instrument 30 is limited in volume and pressure so as not to damage canulated medical instrument 30. As indicated previously, the level of pressure required to deform sleeve 62 and release fluid from pressure/flow regulator 20 depends upon the design and characteristics of sleeve 62. By compounding the material forming sleeve 62 and by designing the inner and outer diameters of sleeve 62 in light of the diameter of cylindrical portion 46 of pressure/flow regulator 20, the release pressure of sleeve 62 can be adjusted.

Sleeve 62 is also designed to limit the flow and pressure to instrument connector end 76 to prevent the release of canulated surgical instrument 30 therefrom. As described above, transverse wall 78 on instrument connector end 76 of instrument connector 26 forms a seal around tubular portion 88 of canulated surgical instrument 30. If the pressure in instrument connector end 76 exceeds a desired value, instrument connector end 76 will expand such that transverse wall 78 no longer seals against tubular portion 88. As a result, canulated surgical instrument 30 may be released from instrument connector end 76. According to the present invention, the pressure within instrument connector end 76 is regulated by apertures 56 in cylindrical portion 46 of pressure/flow regulator 20 and sleeve 62. If the pressure in instrument connector end 76 should exceed a desired valve, the pressure would cause sleeve 62 to deform, separate from pressure/flow regulator 20, and allow fluid to escape therefrom.

It is also contemplated that pressure/flow regulator 20 is used during a drying phase in the medical washer. During the drying phase, dry, heated air is circulated over and through canulated surgical instrument 30. Pressure/flow regulator 20 is designed to limit the flow and pressure of dry, heated air to canulated surgical instrument 30, during the drying phase.

The present invention thus provides a simple and relatively inexpensive method of controlling flow and pressure to a canulated medical instrument in a medical washer so as to prevent damage thereto. The present invention also provides a simple and relatively inexpensive method of maintaining a canulated medical instrument attached to a medical washer.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A pressure/flow regulator, comprising:
a device having an input end that is connectable to a source of fluid, and an output end fluidly connected to a medical instrument, the device having a passage running from the input end to the output end to carry the fluid therethrough; and,
a polymeric sleeve disposed on the device, the polymeric sleeve covering at least one aperture that communicates with the passage, the polymeric sleeve having a resiliency capable of retaining the fluid within the at least one aperture until a predetermined pressure of the fluid within the aperture is attained at which point the sleeve deforms releasing pressure of the fluid within the device and preventing pressure damage to the medical instrument.

2. The regulator of claim 1, wherein the fluid is an antimicrobial fluid.

3. The regulator of claim 1, wherein the medical instrument is canulated.

4. The regulator of claim 1, wherein the sleeve is comprised of ethylene propylene diene monomer.

5. The regulator of claim 1, wherein the regulator has four apertures.

6. The regulator of claim 1, wherein the sleeve deforms at a pressure of about 10 psig to about 15 psig.

7. The regulator of claim 1, further comprising:
an instrument connector for fluidly connecting the regulator to the medical instrument, the connector having a first end, a second end and a bore extending from the first end to the second end.

8. The regulator of claim 7, wherein the instrument connector includes a wall transversing the bore and an opening extending through the wall, the opening being dimensioned to receive the medical instrument such that the wall sealingly engages the medical instrument.
